(19) United States
(12) Patent Application Publication
(10) Pub. No.: US 2020/0069677 A1
(43) Pub. Date: Mar. 5, 2020

(54) MARKERS FOR PERSONALIZED CANCER TREATMENT WITH LSD1 INHIBITORS

(71) Applicant: Constellation Pharmaceuticals, Inc., Cambridge, MA (US)

(72) Inventors: John McGrath, Natick, MA (US); Patrick Trojer, Reading, MA (US)

(73) Assignees: Constellation Pharmaceuticals, Inc., Cambridge, MA (US); Constellation Pharmaceuticals, Inc., Cambridge, MA (US)

(21) Appl. No.: 16/467,396

(22) PCT Filed: Dec. 8, 2017
(86) PCT No.: PCT/US2017/065236

§ 371 Date: Jun. 6, 2019

Related U.S. Application Data
(60) Provisional application No. 62/461,381, filed on Feb. 21, 2017.
(60) Provisional application No. 62/431,893, filed on Dec. 9, 2016.

(51) Int. Cl. A61K 31/495; C12Q 1/6886; A61K 45/06; A61P 35/00; A61K 9/00; A61K 31/445; A61K 31/136; A61K 31/4155; A61K 31/397; A61K 31/506; A61K 31/505; A61K 31/496; A61K 31/40

(52) U.S. Cl. CPC A61K 31/495; C12Q 2600/158; A61K 45/06; A61P 35/00; A61K 9/0053; A61K 31/445; A61K 31/136; A61K 31/4155; A61K 31/397; A61K 31/506; A61K 31/505; A61K 31/496; A61K 31/40; C12Q 2600/112; C12Q 1/6886

(57) ABSTRACT

Provided herein are methods of treating cancers characterized by a high expression of GFIIB, comprising administering a therapeutically effective amount of a KDMIA inhibitor.

### RELATED APPLICATIONS

This application claims priority to U.S. Provisional Application No. 62/431,893, filed Dec. 9, 2016 and U.S. Provisional Application No. 62/461,381, filed Feb. 21, 2017, the contents of each of which are incorporated herein by reference.

### BACKGROUND

Lysine-specific histone demethylase 1A (KDM1A), also known as LSD1, is a protein in humans that in encoded by the KDM1A gene and specifically demethylates mono- or dimethylated histone H3 lysine-4 (H3K4) and H3 lysine-9 (H3K9) via a redox process. See e.g., Biochimica et Biophysica Acta 1829 (2013) 981-986. KDM1A has been found to possess oncogenic properties in several cancers ranging from prostate (Cancer Res., 66 (2006), pp. 11341-11347), bladder (Mol. Carcinog., 50 (2011), pp. 931-944) neuroblastomas, (Cancer Res., 69 (2009), pp. 2065-2071) lung cancers, (PLoS One, 7 (2012), p. e35065) sarcomas and hepato-carcinomas (Tumour Biol. 2013 February; 34(1):173-80), and KDM1A pharmacological inhibitors have been shown to treat e.g., leukemias (Nat. Med., 18 (2012), pp. 605-611) and solid tumors (Tumour Biol. 2013 February; 34(1):173-80). Despite advances toward the development of potent and selective KDM1A inhibitors as anti-cancer therapies, patient populations for which these inhibitors are particularly effective remain unknown.

The development of targeted cancer therapeutics has intensified interest in the identification of biomarkers that have the potential to predict the response of patients to particular targeted therapies, thereby enabling the physician to tailor therapeutic regimens specific to each patient. A previous report has suggested that a DNA hypomethylation signature predicted the anti-tumor activity of KDM1A inhibitors, specifically in small cell lung cancer (SCLC) (Cancer Cell, 28 (2015), pp. 57-69). Additionally, a gene expression signature, consisting of 38 genes identified in SCLC cells, has been reported as having a possible predictive benefit in identifying KDM1A inhibitor responder patients (International Application No.:PCT/EP2016/073821, Publication No. WO/2017/060319 (published: Apr. 13, 2017). Disclosed herein are patient populations that are particularly suited for treatment with a KDM1A inhibitor.

### SUMMARY

GFI1B (Growth Factor Independent 1B) is a zinc-finger containing transcriptional regulator primarily expressed in cells of hematopoietic lineage. GFI1B protein assembles into complexes with numerous other transcriptional regulatory proteins to control expression of genes involved in the development and maturation of erythrocytes and megakaryocytes, as well as the maintenance of hematopoietic stem cells (Blood, 105(2005), pp. 1448-55). GFI1B functions as a transcriptional repressor by recruiting the KDM1A-CoREST-HDAC complex to its target gene promoters to repress genes involved in multilineage blood cell development (Mol Cell, 27(2007), pp. 562-72). Upon recruitment to these target sites, HDACs and KDM1A act to modify chromatin structure by removing the activating acetylation and methylation marks from nearby histones. GFI1B requires this interaction with KDM1A to epigenetically reprogram the hemogenic endothelium (HE) to enable the development of hematopoietic stem cells (HSCs), which give rise to the various blood cell lineages (Nat Cell Biol, 18(2016), pp. 21-32). Also, GFI1B activity has been found to promote malignant transformation and hematological cancer progression (Blood, 126(2015), pp. 2561-2569)

Recent findings have demonstrated that treatment of human leukemia and SCLC cell lines with a KDM1A inhibitor perturbed the interaction between KDM1A and GFI1B (Mol Cancer Ther, 16(2017), pp. 273-284), as well as the association between KDM1A and the transciptional repressor INSM1, specifically in SCLC cells (Cancer Res, 77 (2017), pp. 4652-4662). Example 1 below shows that KDM1A inhibitors abrogate the interaction between KDM1A and GFI1B in a human leukemia cell line model. See also e.g., FIG. 1 and FIG. 3. Human leukemia cell lines expressing higher levels of GFI1B mRNA and protein were found to be significantly more responsive to KDM1A inhibition than cell lines with comparatively low GFI1B expression levels. See e.g., FIG. 4 and FIG. 5. Based on this discovery, disclosed herein are methods of selecting cancer patients which are likely to be responsive to treatment with KDM1A inhibition and then treating said patients with one or more KDM1A inhibitors. Additionally, it is known that acute myeloid leukemia (AML) patients with high levels of GFI1B expression in blast cells have a poor prognosis and an increased risk of relapse (Blood 2013; 122(21):3795). Thus, AML patients with high levels of GFI1B expression are particularly difficult to treat. Disclosed herein are methods to treat this particularly difficult to treat patient population.

### BRIEF DESCRIPTION OF THE FIGURES

FIG. 1 illustrates that treatment of SET2 cells with RN-1 results in displacement of GFI1B from the KDM1A/CoREST complex.

FIG. 2 illustrates that the KDM1A/CoREST complex remains intact in RN-1 treated SET2 cells.

FIG. 3 illustrates that treatment of HEL cells with RN-1 results in disassociation of GFI1B from the KDM1A/CoREST complex.

FIG. 4 illustrates GFI1B expression levels in a leukemia cell line panel.

FIG. 5 correlates leukemia cell lines from FIG. 4 to their respective disease states.

FIG. 6 illustrates GFI1 expression levels in a leukemia cell line panel.

FIG. 7 correlates leukemia cell lines from FIG. 6 to their respective disease states.

FIG. 8 illustrates the correlation between GFI1B expression levels and the response to KDM1A inhibitors.

FIG. 9 illustrates that GFI1B expression levels vary in AML patient samples.

FIG. 10 illustrates that GFI1 expression levels are fairly uniform across AML patient samples.

FIG. 11 illustrates that GFI1B knockdown inhibits growth of SET2 cells.

FIG. 12 illustrates the in vivo efficacy of the KDM1A inhibitor GSK2879552 on SET2 xenografts.

### DETAILED DESCRIPTION

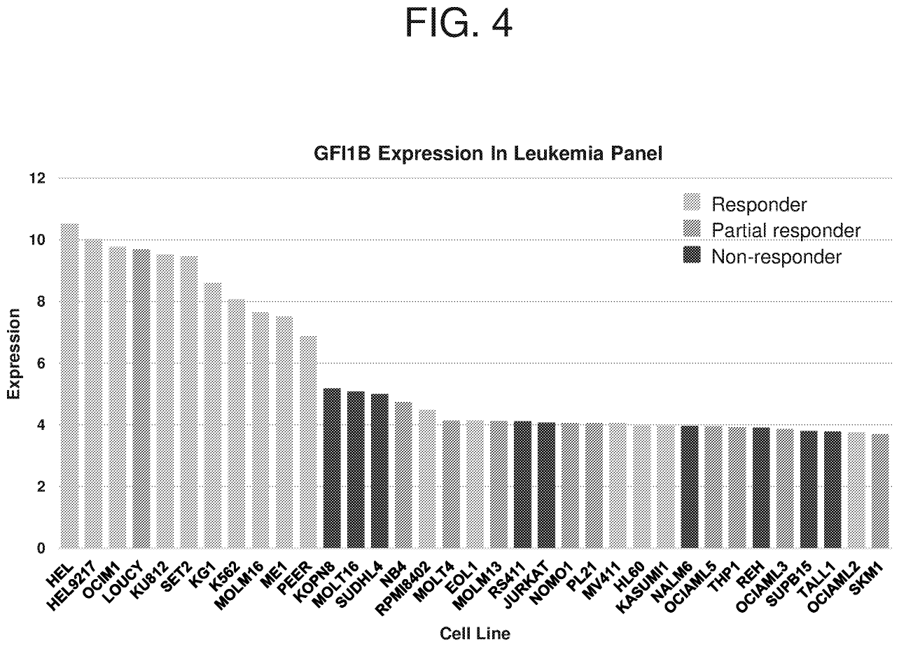

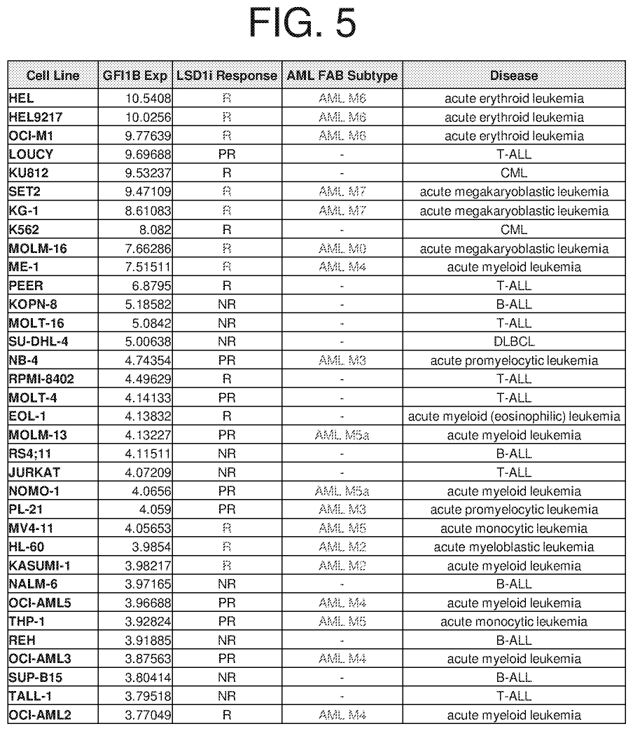

Based in part on the discovery that cell lines expressing higher levels of GFI1B are more responsive to KDM1A inhibition (see e.g., FIG. 4 and FIG. 5), provided herein are methods of selecting patients which are likely to be responsive to treatment with KDM1A inhibition (i.e., those expressing higher levels of GFI1B), and then treating said patients with one or more KDM1A inhibitors. Thus, in one aspect, the present disclosure provides a method of treating a subject with a cancer characterized by a high expression level of GFI1B.

In one aspect, prior to treatment with a therapeutically effective amount of an KDM1A inhibitor, the cancer was determined to exhibit a high expression level of GFI1B. Such a determination can be made by routine diagnostics methods. These methods include, but are not limited to, biopsy, blood tests, and other diagnostic indicators such as peripheral blood mononuclear cells (PBMCs), PBMC subpopulations, circulating blasts (CD34+ cells), circulating tumor cells and circulating exosomes. In one aspect, the step of performing a biopsy of the subject's cancer prior to treatment and determining if the cancer exhibits a high expression level of GFI1B mRNA is by methodology known in the art, for example, by quantitative PCR or GFI1B protein by immunohistochemistry or Western blot. See e.g., RT-qPCR analysis discussed below.

In another aspect, provided herein is a method of treating a subject with a cancer comprising determining the expression level of GFI1B; and administering to the subject a therapeutically effective amount of a KDM1A inhibitor, if the subject's cancer exhibits a high expression level of GFI1B.

In another aspect, provided herein is a method of treating a subject with a cancer comprising taking a tissue sample from the subject's cancer; determining the expression level of GFI1B; and administering to the subject a therapeutically effective amount of a KDM1A inhibitor, if the subject's cancer exhibits a high expression level of GFI1B.

In another aspect, provided herein is a method of predicting the efficacy of a KDM1A inhibitor to treat cancer in patient comprising obtaining a sample from the patient and determining the expression level of GFI1B of the cancer, wherein the KDM1A inhibitor is likely to be effective if the expression level of GFI1B is high.

In another aspect, provided herein is a method of selecting a patient who is likely to respond to treatment with a KDM1A inhibitor, said method comprising determining the expression level of GFI1B of a cancer of the patient, wherein the patient is likely to respond to treatment if the expression level of GFI1B is high.

In one aspect, the “expression level” may be normalized to one or more comparator markers in order to make it comparable across samples. In one aspect, the comparator may be a housekeeping protein or gene used as a standard control for normalization, such as, for example, β-actin (ACTB), Glyceraldehyde-3-Phosphate Dehydrogenase (GAPDH), or TATA-box Binding Protein (TBP).

In another aspect, provided herein is a method of treating a subject with a cancer, comprising determining the expression level of GFI1B of the cancer and administering to the subject a therapeutically effective amount of a cancer therapy other than the administration of a KDM1A inhibitor, if the expression level of GFI1B of the subject's cancer is not high; and administering a therapeutically effective amount of a KDM1A inhibitor, if the expression level of GFI1B is high. In certain instances, treatment effects from the use of KDM1A inhibitors on certain subject's cancers can be improved by combination therapies with other anti-cancer agents. For example, subjects who had low response rates to treatment with a KDM1A inhibitor as the sole active agent can have higher response rates when administered an effective amount of a KDM1A inhibitor and an additional anti-cancer agent. Combination treatment can also be used for subject who have high expression levels of GFI1B.

In one aspect, provided herein are methods of treating a cancer in a subject, comprising administering to the subject an effective amount of a KDM1A inhibitor and an anti-cancer agent. In one aspect, the cancer treated by this combination treatment is characterized by an expression level of GFI1B defined by an expression value falling at or below 75%, or e.g., between the 1^{st} to 75^{th} percentile, 25^{th} to 75^{th} percentile, between the 30^{th} to 70^{th} percentile, between the 35^{th} to 65 percentile, between the 25^{th} to 50^{th} percentile, the 25^{th} percentile, the 30^{th} percentile, the 35^{th} percentile the 40^{th} percentile, the 45^{th} percentile, or the 50^{th} percentile of measured values from tissue samples obtained from the same cancer type in a population of subjects. In one aspect, the cancer was determined to comprise an expression level of GF1B defined by a an expression value falling at or below 75%, or e.g., between the 1^{st} to 75^{th} percentile, 25^{th} to 75^{th} percentile, between the 30^{th} to 70^{th} percentile, between the 35^{th} to 65 percentile, between the 25^{th} to 50^{th} percentile, the 25^{th} percentile, the 30^{th} percentile, the 35^{th} percentile the 40^{th} percentile, the 45^{th} percentile, or the 50^{th} percentile of measured values from tissue samples obtained from the same cancer type in a population of subjects, prior to treatment an effective amount of a KDM1A inhibitor and an anti-cancer agent. In one aspect, the tissue sample are taken from tumors.

In one aspect, cancer therapies other than a KDM1A inhibitor include, but are not limited to, surgery, radiation therapy, immunotherapy, endocrine therapy, gene therapy and administration of an anti-cancer agent other than a KDM1A inhibitor. In another aspect, cancer therapies other than a KDM1A inhibitor include, but are not limited to, surgery, radiation therapy, immunotherapy, endocrine therapy, gene therapy, and epigenetic therapy, including the administration of an agent other than a KDM1A inhibitor.

Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body. Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy. In addition, immunotherapy drugs referred to as immune checkpoint inhibitors are designed to unshackle the patient's own immune system cells from attacking tumor cells. Examples include the drugs nivolumab (Opdivo) and pembrolizumab (Keytruda), which are monoclonal antibodies recognizing the PD-1 antigen, approved for the treatment of advanced classical Hodgkin lymphoma; atezolizumab (Tecentriq), a fully humanized monoclonal antibody against the protein programmed cell death-ligand 1 (PD-L1), approved for bladder cancer treatment; and ipilimumab (Yervoy), which is a monoclonal antibody that activates the immune system by targeting the CTLA-4 protein.

Examples of passive immunotherapies include but are not limited to monoclonal antibodies and targeted therapies containing toxins. Monoclonal antibodies include naked antibodies and conjugated monoclonal antibodies (also called tagged, labeled, or loaded antibodies). Naked monoclonal antibodies do not have a drug or radioactive material attached whereas conjugated monoclonal antibodies are joined to, for example, a chemotherapy drug (chemolabeled), a radioactive particle (radiolabeled), or a toxin (immunotoxin). Examples of these naked monoclonal antibody drugs include, but are not limited to Rituximab (Rituxan), an antibody against the CD20 antigen used to treat, for example, B cell non-Hodgkin lymphoma; Trastuzumab (Herceptin), an antibody against the HER2 protein used to treat, for example, advanced breast cancer; Alemtuzumab (Campath), an antibody against the CD52 antigen used to treat, for example, B cell chronic lymphocytic leukemia (B-CLL); Cetuximab (Erbitux), an antibody against the EGFR protein used, for example, in combination with irinotecan to treat, for example, advanced colorectal cancer and head and neck cancers; and Bevacizumab (Avastin) which is an antiangiogenesis therapy that works against the VEGF protein and is used, for example, in combination with chemotherapy to treat, for example, metastatic colorectal cancer. Examples of the conjugated monoclonal antibodies include, but are not limited to radiolabeled antibody Ibritumomab tiuxetan (Zevalin), a monoclonal antibody against the CD20 antigen which delivers radioactivity directly to cancerous B lymphocytes and is used to treat, for example, B cell non-Hodgkin lymphoma; radiolabeled antibody Tositumomab (Bexxar), another monoclonal antibody recognizing the CD20 antigen, which is used to treat, for example, certain types of non-Hodgkin lymphoma; and immunotoxin Gemtuzumab ozogamicin (Mylotarg), a monoclonal antibody to CD33 linked to the cytotoxic agent calicheamicin and is used to treat, for example, acute myelogenous leukemia (AML). BL22 is a conjugated monoclonal antibody for treating, for example, hairy cell leukemia, immunotoxins for treating, for example, leukemias, lymphomas, and brain tumors, and radiolabeled antibodies.

An additional example of passive immunotherapy, involving gene therapy, would include CAR (Chimeric antigen receptor) T-cell therapy, which involves genetically modifying the patient's own T cells to target and enhance their cancer-fighting ability. FDA approved CAR-T therapies include axicabtagene ciloleucel (Yescarta), which targets the CD19 antigen and is approved for the treatment of diffuse large B-cell lymphoma; and tisagenlecleucel (Kymriah), used for the treatment of relapsed/refractory B-cell precursor acute lymphoblastic leukemia.

In one aspect, immunotherapies that can be used in the present teachings include adjuvant immunotherapies. Examples include cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); and combinations thereof, such as, for example, combinations of, interleukins, for example, IL-2 with other cytokines, such as IFN-alpha.

An endocrine therapy is a treatment that adds, blocks or removes hormones. For example, chemotherapeutic agents that can block the production or activity of estrogen have been used for treating breast cancer. In addition, hormonal stimulation of the immune system has been used to treat specific cancers, such as renal cell carcinoma and melanoma. In one embodiment, the endocrine therapy comprises administration of natural hormones, synthetic hormones or other synthetic molecules that may block or increase the production or activity of the body's natural hormones. In another embodiment, the endocrine therapy includes removal of a gland that makes a certain hormone.

A gene therapy is the insertion of genes into a subject's cell and biological tissues to treat diseases, such as cancer. Exemplary gene therapy includes, but is not limited to, a germ line gene therapy and a somatic gene therapy, including the genetic modification of patient-derived immune T-cells referred to as CAR-T cell therapy.

In one aspect, cancer therapies other than a KDM1A inhibitor are other anti-cancer agents. An “anti-cancer agent” is a compound, which when administered in an effective amount to a subject with cancer, can achieve, partially or substantially, one or more of the following: arresting the growth, reducing the extent of a cancer (e.g., reducing size of a tumor), inhibiting the growth rate of a cancer, and ameliorating or improving a clinical symptom or indicator associated with a cancer (such as tissue or serum components), or increasing longevity of the subject.

The anti-cancer agent suitable for use in the methods described herein include anti-cancer agents that have been approved for the treatment of cancer. In one aspect, the anti-cancer agent includes, but is not limited to, a targeted antibody, an immune checkpoint inhibitor, an angiogenisis inhibitor, an epigenetic agent, an alkylating agent, an antimetabolite, a vinca alkaloid, a taxane, a podophyllotoxin, a topoisomerase inhibitor, a hormonal antineoplastic agent and other antineoplastic agents.

Examples of alkylating agents useful in the methods of the present teachings include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful in the methods of the present teachings include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of plant alkaloids and terpenoids or derivatives thereof include, but are not limited to, vinca alkaloids (e.g., vincristine, vinblastine, vinorelbine, vindesine), podophyllotoxin, and taxanes (e.g., paclitaxel, docetaxel). Examples of a topoisomerase inhibitor includes, but is not limited to, irinotecan, topotecan, amsacrine, etoposide, etoposide phosphate and teniposide. Examples of antineoplastic agents include, but are not limited to, actinomycin, anthracyclines (e.g., doxorubicin, daunorubicin, valrubicin, idarubicin, epirubicin), bleomycin, plicamycin and mitomycin.

In one aspect, the anti-cancer agents that can be used in the present teachings include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents/drugs that can be used in the present teachings include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In one aspect, cancer therapies are anti-cancer agents suitable for treating leukemias. Exemplary treatments include, but are not limited to, Abitrexate® (Methotrexate), Arranon® (Nelarabine), Asparaginase Erwinia chrysanthemi, Blinatumomab, Blincyto® (Blinatumomab), Cerubidine® (Daunorubicin Hydrochloride), Clafen® (Cyclophosphamide), Clofarabine®, Clofarex® (Clofarabine), Clolar® (Clofarabine), Cyclophosphamide, Cytarabine, Cytosar-U® (Cytarabine), Cytoxan® (Cyclophosphamide), Dasatinib, Daunorubicin Hydrochloride, Doxorubicin Hydrochloride, Erwinaze® (Asparaginase Erwinia Chrysanthemi), Folex® (Methotrexate), Folex PFS® (Methotrexate), Gleevec® (Imatinib Mesylate), Iclusig® (Ponatinib Hydrochloride), Imatinib Mesylate, Marqibo® (Vincristine Sulfate Liposome), Mercaptopurine, Methotrexate, Methotrexate LPF® (Methorexate), Mexate® (Methotrexate), Mexate-AQ® (Methotrexate), Nelarabine, Neosar® (Cyclophosphamide), Oncaspar® (Pegaspargase), Pegaspargase, Ponatinib Hydrochloride, Prednisone, Purinethol® (Mercaptopurine), Purixan® (Mercaptopurine), Rubidomycin® (Daunorubicin Hydrochloride), Spryce®l (Dasatinib), Tarabine PFS® (Cytarabine), Vincasar PFS® (Vincristine Sulfate), Vincristine Sulfate, Vincristine Sulfate Liposome, Hyper-CVAD, Arsenic Trioxide, Idamycin (Idarubicin Hydrochloride), Idarubicin Hydrochloride, Mitoxantrone Hydrochloride, Tabloid (Thioguanine), Thioguanine, Trisenox® (Arsenic Trioxide), Alemtuzumab, Ambochlorin® (Chlorambucil), Arzerra® (Ofatumumab), Bendamustine Hydrochloride, Campath® (Alemtuzumab), Chlorambucil, Fludara® (Fludarabine Phosphate), Fludarabine Phosphate, Gazyva® (Obinutuzumab), Ibrutinib, Idelalisib, Imbruvica® (Ibrutinib), Leukeran® (Chlorambucil), Linfolizin® (Chlorambucil), Mechlorethamine Hydrochloride, Mustargen® (Mechlorethamine Hydrochloride), Obinutuzumab, Ofatumumab, Rituxan® (Rituximab), Rituximab, Treanda® (Bendamustine Hydrochloride), Venclexta® (Venetoclax), Venetoclax, Zydelig® (Idelalisib), chlorambucil-prednisone, CVP, Bosulif (Bosutinib), Bosutinib, Busulfan, Busulfex (Busulfan), Hydrea® (Hydroxyurea), Hydroxyurea, Mechlorethamine Hydrochloride, Myleran® (Busulfan), Neosar (Cyclophosphamide), Nilotinib, Omacetaxine Mepesuccinate, Synribo® (Omacetaxine Mepesuccinate), and Tasigna® (Nilotinib).

As used herein “high expression” means an expression value equivalent to the top quartile of the measured expression values. “Top quartile” can be obtained by collecting expression values of GFI1B from the cancers (e.g., tissue samples) of a population of subjects, e.g., at least 25 subjects, at least 50 subjects, at least 100 subjects, at least 500 subjects, at least 1000 subjects or the like, having the same cancers and then assessing whether the expression value of a new subject falls within the top 25% quartile. The expression value can be, for example, the level of GFI1B mRNA, which can be determined as described in the RT-qPCR analysis in the materials and methods section. In one aspect, “high expression” means a GFI1B mRNA expression level of 6 or greater, as determined on an Affymetrix microarray platform with the expression level defined as the gene-centric RMA-normalized log 2 ratio expression value.

In an alternative, “high expression” refers to a level of GFI1B mRNA or protein in the sample from the individual or patient above a defined reference level or to an overall increase of 5%, 10%, 20% 25% 30% 40%, 50%, 60%, 70%, 80%, 85%, 90%, 95%, 100% or greater, determined by the methods described herein, as compared to the reference level.

The “reference level” refers to the median normalized GFI1B expression determined in reference samples from healthy individuals with the reference sample being from essentially the same type of cells, tissue, organ or body fluid source as the sample from the individual or patient subjected to the method of the invention. In one aspect, the reference level can be determined in reference samples from a population of patients with the same neoplastic disease affecting the patient.

In one aspect, the reference level can be obtained by deternining the average normalized RNA expression [a value which can be in any form of mRNA expression measurement, such as, e.g., expression levels derived from RNA-sequencing such as normalized read counts and RPKM (Reads per Kilobase of Million mapped reads) or normalized cycle threshold (Ct) values from RT-PCR measurements] for GFI1B between two human leukemia cell lines, CL_{R} and CL_{NR}, wherein CL_{R} is a responder or sensitive cell line with the highest expression of GFI1B mRNA, and CL_{NR} is a non-responder or insensitive/resistant cell line with the lowest expression of the GFI1B gene.

In one aspect, the reference level can be obtained by determining the average normalized GFI1B protein level between two human leukemia cell lines, CL_{R} and CL_{NR}, wherein CL_{R} is a responder or sensitive cell line with the highest levels of GFI1B protein, and CL_{NR} is a non-responder or insensitive/resistant cell line with the lowest expression of the GFI1B protein.

In one aspect, the reference level can, e.g., be set to any percentage between 25% and 75% of the overall distribution of the values in a disease entity being investigated. In other embodiments the reference level can, e.g., be set to the median, tertiles or quartiles as determined from the overall distribution of the values in reference samples from the disease entity being investigated. The reference level may vary depending on various physiological parameters such as age, gender or subpopulation, as well as on the means used for the determination of the GFI1B expression levels.

KDM1A or LSD1 inhibitors described herein include e.g., small molecules that are capable of inhibiting (K)-specific demethylase 1A (LSD1) activity. Inhibition can be measured in vitro, in vivo, or from a combination thereof. In one aspect, the KDM1A inhibitors in the methods described herein include, but are not limited to, those described in WO2016172496, WO 20170267678, WO 2017079670, WO 2016130952, WO 2017114497, WO 2017149463, WO 2016007736, U.S. Pat. No. 8,853,408, WO 2011131697, WO 2012013727, WO 2012013728, WO 2012107498, WO 2012107499, WO 2013033688, WO 2012150042, WO 2013143597, WO 2012052390, US 2013210888, WO 2012047852, WO 2012034116, US 2012322877, US 2013197088, US 2012108648, WO 2012071469, US 2013095067, WO 2012156537, WO 2012156531, WO 2012072713, WO 2013022047, US 2013137720, US 2012142784, WO 2013025805, US 2013123344, PCT/US2017/058405, PCT/US2017/058404, CN 103054869, and US 20160009721, as well as

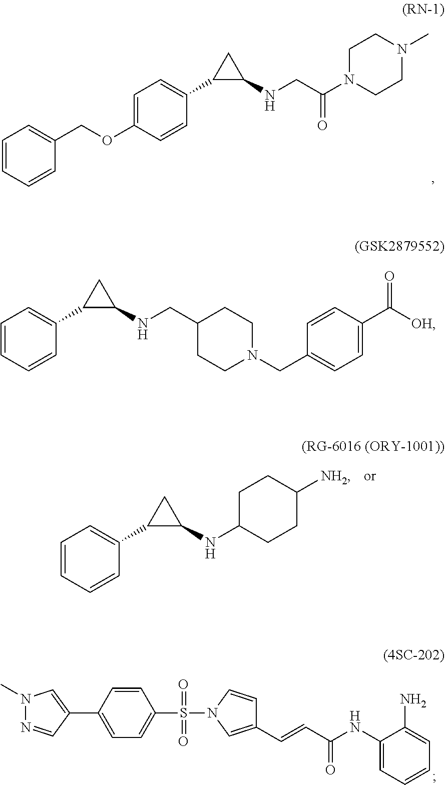

or a pharmaceutically acceptable salt thereof.

In one aspect, the KDM1A inhibitors in the methods described herein are

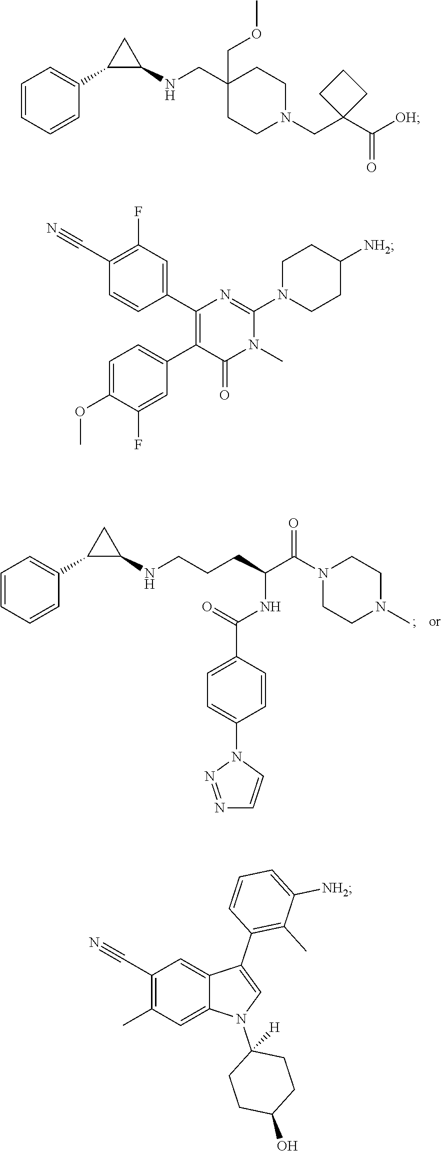 [SMILES: COCC1(CN[C@@H]2C[C@H]2c2ccccc2)CCN(CC2(C(=O)O)CCC2)CC1 ; COc1ccc(-c2c(-c3ccc(C#N)c(F)c3)nc(N3CCC(N)CC3)n(C)c2=O)cc1F ; CN1CCN(C(=O)[C@H](CCCN[C@@H]2C[C@H]2c2ccccc2)NC(=O)c2ccc(-n3ccnn3)cc2)CC1 ; Cc1cc2c(cc1C#N)c(-c1cccc(N)c1C)cn2[C@H]1CC[C@@H](O)CC1]

or a pharmaceutically acceptable salt thereof.

In one aspect, the KDM1A inhibitors in the methods described herein are

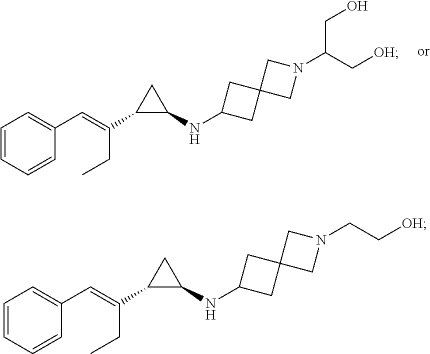 [SMILES: CC/C(=C\c1ccccc1)[C@@H]1C[C@H]1NC1CC2(C1)CN(C(CO)CO)C2 ; "or" ; CC/C(=C\c1ccccc1)[C@@H]1C[C@H]1NC1CC2(C1)CN(CCO)C2]

or a pharmaceutically acceptable salt thereof. In another aspect, the KDM1A inhibitors in the methods described herein are

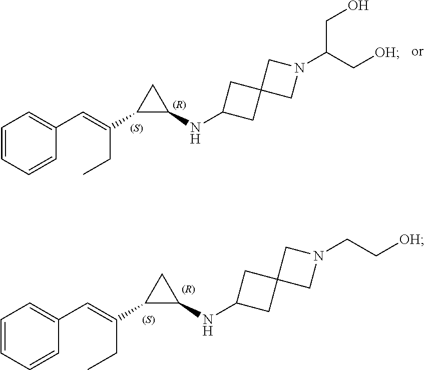

or a pharmaceutically acceptable salt thereof. In another aspect, the KDM1A inhibitor in the methods described herein is

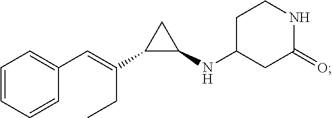 [SMILES: CC/C(=C\c1ccccc1)[C@@H]1C[C@H]1NC1CCNC(=O)C1]

or a pharmaceutically acceptable salt thereof. In another aspect, the KDM1A inhibitor in the methods described herein is

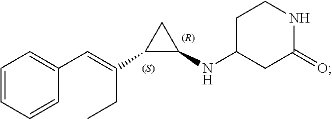

or a pharmaceutically acceptable salt thereof. In another aspect, the KDM1A inhibitor in the methods described herein is

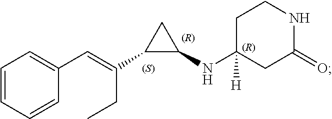

or a pharmaceutically acceptable salt thereof. In another aspect, the KDM1A inhibitor in the methods described herein is

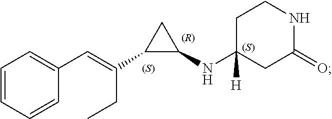

or a pharmaceutically acceptable salt thereof.

When the configuration of two or more substituents about a double bond in the KDM1A inhibitors described herein is indicated by structure; by “E” or “Z” designations; or by “cis” or “trans”; the depicted stereochemical purity with respect to that double bond is at least 85%, at least 90%, at least 95%, at least 97%, at least 98% or at least 99% by weight. Stereochemical purity by weight with respect to a double bond means the percent by weight of the KMD1 inhibitor in a composition having the indicated stereochemistry about the double bond. For example, when the double bond is represented by

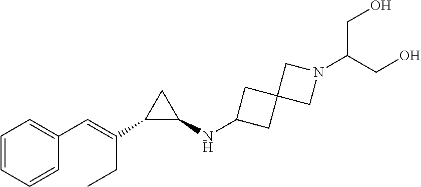 [SMILES: CC/C(=C\c1ccccc1)[C@@H]1C[C@H]1NC1CC2(C1)CN(C(CO)CO)C2]

the KMD1 inhibitor has a stereochemical purity with respect to the depicted trans (E) stereochemistry about the double bond, i.e., at least 85%, at least 90%, at least 95%, at least 97%, at least 98% or at least 99% by weight of the KMD1 inhibitor in a composition contains the represented trans (i.e., E) double bond.

When the stereochemistry about the cyclopropyl in the KDM1A inhibitors described herein is indicated by structure only, the structure is meant to depict the relative stereochemistry at one of the chiral centers in the cyclopropyl relative to the stereochemistry at the other chiral center, and not the absolute stereochemistry at either chiral center in the cyclopropyl. For example, when the stereochemistry about the cyclopropyl is depicted by structure only as being trans, the stereochemical purity of the compound with respect to the depicted trans configuration about the cyclopropyl is at least 85%, at least 90%, at least 95%, at least 97%, at least 98% or at least 99% by weight, i.e., the percent by weight of the KMD1 inhibitor in a composition having the trans stereochemistry at the cyclopropyl is at least 85%, at least 90%, at least 95%, at least 97%, at least 98% or at least 99% by weight. For example, the KMD1 inhibitor represented by the formula:

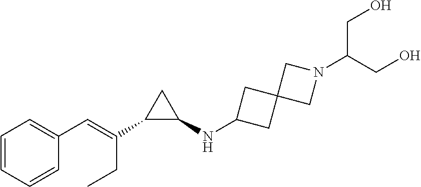 [SMILES: CC/C(=C\c1ccccc1)[C@@H]1C[C@H]1NC1CC2(C1)CN(C(CO)CO)C2]

means that at least 85%, at least 90%, at least 95%, at least 97%, at least 98% or at least 99% by weight of the KMD1 inhibitor in a composition has the depicted trans configuration about the cyclopropyl; at least at least 85%, at least 90%, at least 95%, at least 97%, at least 98% or at least 99% by weight of the KMD1 inhibitor in the composition contains the other trans configuration as:

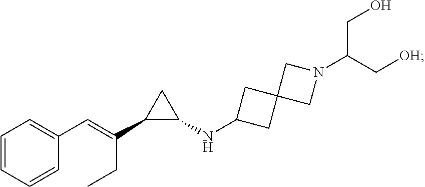 [SMILES: CC/C(=C\c1ccccc1)[C@H]1C[C@@H]1NC1CC2(C1)CN(C(CO)CO)C2]

or at least 85%, at least 90%, at least 95%, at least 97%, at least 98% or at least 99% by weight of the compound in a composition is a mixture of the two trans configurations.

When the absolute stereochemistry of chiral centers in a KMD1 inhibitor of the methods described herein are indicated structurally and by “R” or “S” designations, it is to be understood that the depiction means the depicted stereoisomer at a stereochemical purity of at least 85%, at least 90%, at least 95%, at least 97%, at least 98% or at least 99% by weight, i.e., the percent by weight of the indicated stereoisomer of the KMD1 inhibitor represented in a composition. For example, the KMD1 inhibitor represented by the formula:

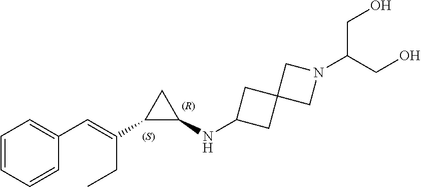

means at least 85%, at least 90%, at least 95%, at least 97%, at least 98% or at least 99% by weight of the KMD1 inhibitor in the composition contains of the depicted stereoisomer. When the structure being depicted by structure and by “R” or “S” designation is a single enantiomer, the enantiomeric purity is at least 95% (e.g., at least 96%, at least 97%, at least 98%, at least 99%, at least 99.5%, or at least 99.9%).

The 1- and 2-positions of the cyclopropyl ring represent the following:

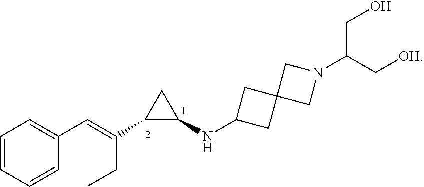 [SMILES: CC/C(=C\c1ccccc1)[C@@H]1C[C@H]1NC1CC2(C1)CN(C(CO)CO)C2]

Unless otherwise specified, when only some of the stereochemical centers in a KMD1 inhibitor are depicted or named by structure, the named or depicted configuration is enriched relative to the remaining configurations, for example, by a molar excess of at least 60%, 70%, 80%, 90%, 99% or 99.9%. For example,

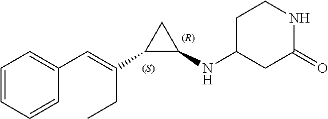

means that that the configuration about the cyclopropyl is stereochemically enriched as 1R, 2S (e.g., by a molar excess of at least 60%, 70%, 80%, 90%, 99% or 99.9%) and that the geometry about the piperidin-2-one may be R or S, or a mixture thereof.

When a compound is depicted structurally without indicating the stereochemistry at a chiral center, it is to be understood that the structure includes either configuration at the chiral center or, alternatively, any mixture of configurations at the chiral center stereoisomers.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, or inhibiting the progress of a cancer, or one or more symptoms thereof, as described herein. Exemplary types of cancer include e.g., Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, neuroendocrine tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma, small cell neuroendocrine carcinomas and carcinoid tumors), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, diffuse intrinsic pontine glioma (DIPG), congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), fallopian tubes (carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma)), Hematologic: myeloid leukemia (acute and chronic), acute lymphoblastic leukemia, chronic lymphocytic leukemia, myeloproliferative diseases (primary myelofibrosis, polycythemia vera, essential thrombocythemia), multiple myeloma, myelodysplastic syndrome, Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, Merkel cell carcinoma, lipoma, angioma, dermatofibroma, keloids, psoriasis; and Adrenal glands: neuroblastoma.

In one aspect, the cancer characterized by a high expression of GFI1B is selected from breast cancer, colorectal cancer, pancreatic cancer, cervical cancer, T cell lymphoma, uveal melanoma, gastric carcinoma, colorectal carcinoma, ovarian carcinoma, hepatocellular carcinoma, melanoma, glioma, cardiac, gastrointestinal, genitourinary tract, liver, bone, nervous system, gynecological, hematologic, skin, and adrenal cancers.

In another aspect, the cancer characterized by a high expression of GFI1B is selected from angiosarcoma, fibrosarcoma, rhabdomyosarcoma, liposarcoma, myxoma, rhabdomyoma, fibroma, lipoma, teratoma, squamous cell carcinoma, undifferentiated small cell carcinoma, undifferentiated large cell carcinoma, adenocarcinoma, alveolar carcinoma, bronchial adenoma, sarcoma, lymphoma, chondromatous hamartoma, mesothelioma, leiomyosarcoma, carcinoma, ductal adenocarcinoma, insulinoma, glucagonoma, gastrinoma, carcinoid tumors, vipoma, carcinoid tumors, Kaposi's sarcoma, leiomyoma, Merkel cell carcinoma, hemangioma, lipoma, neurofibroma, fibroma, tubular adenoma, villous adenoma, hamartoma, Wilm's tumor, transitional cell carcinoma, seminoma, teratoma, embryonal carcinoma, teratocarcinoma, choriocarcinoma, interstitial cell carcinoma, fibroma, fibroadenoma, adenomatoid tumors, lipoma, hepatocellular carcinoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, reticulum cell sarcoma, multiple myeloma, malignant giant cell tumor chordoma, osteochronfroma, benign chondroma, chondroblastoma, chondromyxofibroma, osteoid osteoma, giant cell tumors, osteoma, hemangioma, granuloma, xanthoma, osteitis deformans, meningioma, meningiosarcoma, gliomatosis, astrocytoma, medulloblastoma, glioma, ependymoma, germinoma, glioblastoma multiform, oligodendroglioma, schwannoma, retinoblastoma, congenital tumors, spinal cord neurofibroma, meningioma, endometrial carcinoma, cervical carcinoma, pre-tumor cervical dysplasia, ovarian carcinoma, granulosa-thecal cell tumors, Sertoli-Leydig cell tumors, intraepithelial carcinoma, clear cell carcinoma, squamous cell carcinoma, acute myeloid leukemia, chronic myeloid leukemia, acute lymphoblastic leukemia, chronic lymphocytic leukemia, myeloproliferative diseases, myelodysplastic syndrome, Hodgkin's disease, non-Hodgkin's lymphoma (malignant lymphoma), and neuroblastoma.

In another aspect, the cancer characterized by a high expression of GFI1B is selected from small cell lung carcinoma, neuroblastoma, Merkel cell carcinoma, myeloproliferative diseases, and myelodysplastic syndrome.

In another aspect, the cancer characterized by a high expression level of GFI1B is acute myeloid leukemia or chronic myeloid leukemia.

In another aspect, the cancer characterized by a high expression level of GFI1B is acute erythroid leukemia, acute megakaryoblastic leukemia, T-cell acute lymphoblastic leukaemia, chronic myeloid leukemia, acute promyelocytic leukemia, acute myeloblastic leukemia, or acute monocytic leukemia.

The term “pharmaceutically acceptable carrier, adjuvant, or vehicle” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a KDM1A inhibitor described herein in the composition will also depend upon the particular compound in the composition.

### EXEMPLIFICATION

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

RN-1 was prepared according to the procedures described in McGrath, J. P., et al (2016). Pharmacological inhibition of the histone lysine demethylase KDM1A suppresses the growth of multiple acute myeloid leukemia subtypes. Cancer Res. 76(7):1975-88.

Materials and Methods

### Protein Expression and Purification

Full length N-terminal Hexa-histidine (His)-tagged lysine (K)-specific demethylase 1A cDNA (His-KDM1A) (1-852aa) was purchased from GeneCopoeia (Rockville, Md.) in the pReceiver-B01 vector (Cat # E3231). The plasmid was transformed into BL21 (DE3) competent cells and grown in LB media at 37° C. to an OD₆₀₀ of 0.6. The expression of the recombinant protein was induced by the addition of 0.25 mM IPTG and cultures were grown for 18 hours at 20° C. His-tagged protein was purified using an AKTA Chromatography System with a HisTrap-FF column (GE Healthcare, Marlborough, Mass.). The column was equilibrated using Binding buffer (50 mM NaH2PO4 pH 7.4, 500 mM NaCl, 0.5 mM TCEP, 20 mM imidazole). After protein binding the column was rinsed using binding buffer (30 mM imidazole) and the recombinant protein was eluted in the presence of 250 mM imidazole. Eluted fractions containing KDM1A were combined and dialyzed against dialysis buffer (50 mM Tris-HCl pH 7.5, 250 mM NaCl, 0.5 mM TCEP). The dialyzed protein was concentrated to 1 ml and loaded onto a Superdex S-200 (10/300GL, flow-rate of 0.5 ml/min), previously equilibrated in dialysis buffer. Fractions containing KDM1A were pooled, concentrated to 2-3 mg/ml and stored at −80° C. KDM1B cDNA was cloned into the pDEST20 plasmid (Invitrogen) using Gateway technology and subsequently transfected into Sf9 cells. The N-terminal GST-tagged KDM1B was purified from the Sf9 cell lysates on GST-FF Sepharose beads (GE Healthcare) and eluted by reduced glutathione, pH 8.0 in accordance with the manufacturer's protocol.

### Demethylase Assay

Synthetic Histone 3 lysine 4 dimethyl peptide (H3K4me2) (aa 1-21) was purchased from New England Peptide (Gardner, Mass.). Flavin adenine dinucleotide (FAD) was obtained from Sigma (Cat # F6625). A reaction mixture containing either 4 nM purified His-KDM1A or 8 nM purified GST-KDM1B in assay buffer (50 mM Tris-HCl pH 8.25, 0.01% Triton X-100, 0.005% BSA, 0.25 mM TCEP) and an initiation mixture (5 nM FAD and 4 μM H3K4me2 peptide) were prepared. The reaction was initiated by combining the two solutions and incubating for 40 minutes at 22° C. The reaction products were analyzed by RapidFire High-throughput Mass Spectrometry (Agilent Technologies, Inc., Wakefield, Mass.) to quantitate the demethylated peptide species.

### Cell Lines

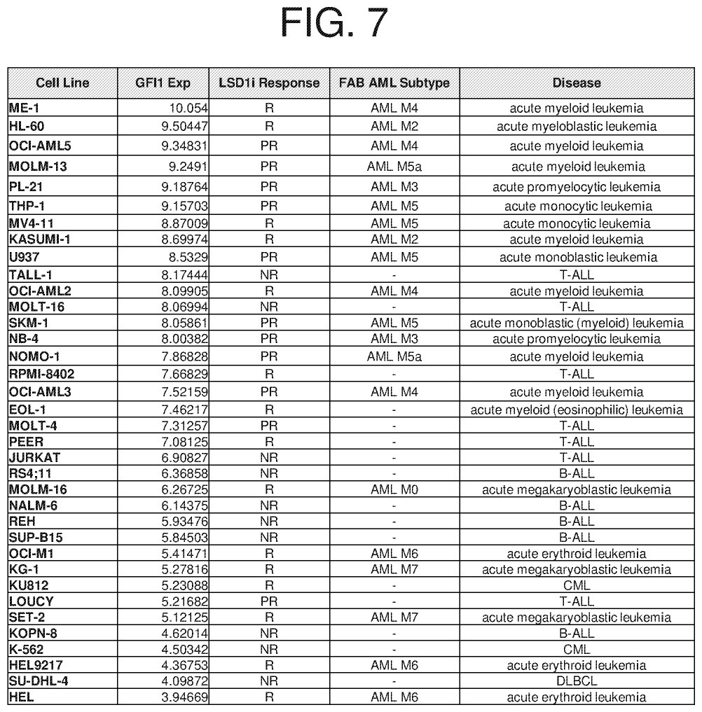

A collection of hematologic cell lines investigated in this study are summarized in FIG. 5 and FIG. 7. The cell lines were obtained either through the ATCC (Manassas, Va.) or DSMZ (Braunschweig, Germany). Cell lines were cultured according to the instructions provided by the respective repositories.

### Antibodies

Antibodies against KDM1A were purchased from Cell Signaling Technologies (CST, #2184) for Western blots and Bethyl Laboratories (A300-215A) for immunoprecipitation. Antibodies recognizing histones and histone modifications included anti-histone H3 (CST, #3638), anti-dimethyl H3K4 (Millipore, 07-030), anti-trimethyl H3K4 (Abcam, ab8580), and anti-dimethyl H3K9 (Abcam, ab1220). Additional antibodies recognizing RCOR1 (Millipore. 07-455), PHF21A (Bethyl, A303-603A), HMG20B (Bethyl, A301-097A), vinculin (Sigma, V9264) and IgG (Abcam, ab46540) were used according to vendor's directions. DyLight™ conjugated goat anti-mouse (ThermoFisher, 35518) and anti-rabbit (ThermoFisher, 35571) IgG secondary antibodies were used for Western blot detection on an Odyssey® CLx Infrared Imaging System (LI-COR Biotechnology, Lincoln, Nebr.).

### Cell Proliferation Assays

Cells were plated at 5,000 cells per well in 96-well tissue culture dishes containing tool compounds arrayed in a 10-point dose curve, ranging from 0 to 10 μM with 4-fold dilutions, and split every fourth day at a ratio to re-establish 5000 cells/well density for DMSO-treated controls. Relative viable cell numbers were assessed by Cell Titer-Glo luminescent cell viability assay (Promega) using an EnVision® Multilabel Plate Reader (Perkin Elmer, Waltham, Mass.). GraphPad Prism 6 (GraphPad Software, Inc., La Jolla, Calif.) was used for curve fitting and determination of GI₅₀ values.

### Immunoprecipitation

SET2 cells growing in RPMI medium supplemented with 20% fetal bovine serum were treated for 24 hours with either 1 μM RN-1 or DMSO as a control. Cells were harvested, washed twice with cold PBS and resuspended in 5 volumes of Buffer A (10 mM Tris-HCL (pH 8.0), 1.5 mM MgCl₂, 10 mM KCl, 0.5 mM DTT, supplemented with protease inhibitors). After 15 minutes on ice, cells were spun down at 1000×g for 15 minutes and the supernatant was removed as the cytosolic fraction. Pellets were resuspended in Buffer C (20 mM Tris-HCl, (pH 7.9), 25% glycerol, 0.42 M NaCl, 1.5 mM MgCl₂, 0.2 mM EDTA, 0.5 mM DTT and protease inhibitors) and sonicated briefly (3 seconds, 40% output). After incubating on ice for 30 minutes, cells were spun at 20,000×g for 20 minutes to pellet the insoluble material. The supernatant was removed (soluble nuclear fraction) and combined with the cytosolic fraction to reconstitute a whole cell lysate with a final NaCl concentration of <200 mM.

For immunoprecipitation, 3 μg of anti-KDM1A antibody (Bethyl Labs, Montgomery, Tex.) or rabbit IgG (Cell Signaling, Danvers, Mass.) were added to 0.5 mg of reconstituted whole cell lysate and placed on a rotator at 4° C. overnight. To isolate the immune complexes, 50 μL of pre-washed Protein G magnetic beads (Dynabeads, Invitrogen) equilibrated in A+C buffer (1:1) were added and the samples were rotated for 4° C. for an additional 2 hours. Beads were captured using a magnetic stand (DynaMag, Invitrogen) and washed 3 times with buffer A+C. The beads were re-suspended in 25 μl 2×SDS loading buffer and resolved on 4-12% SDS-PAGE gels. Co-precipitated proteins were detected by western blotting using the indicated primary antibodies.

### RNAi Knockdown

Stable knockdown of KDM1A and GFI1B was achieved using lentiviral-based shRNA vectors (Cellecta and Sigma). Production and processing of lentiviral stocks were carried out following standard protocols. A set of two non-overlapping gene-specific shRNAs and a non-targeting control shRNA (NTC) shRNA or shRNA targeting luciferase (LUC) were selected for use in all experiments. Cells were transduced using lentiviral vectors expressing the shRNAs at an MOI of 2 using a spin-infection protocol wherein virus was added to 1×10⁶ cells in each well of a 6-well dish in media supplemented with 8 ug/mL Polybrene (Boston BioProducts) and cultures were centrifuged at 1000×g for 1 hour. Media was changed 48 hours later with the addition of puromycin (2 μg/ml). For cells under puromycin selection, after 72 hours cells were plated at equal densities and monitored for cell proliferation via Cell Titer-Glo (CTG, Promega) every 4 days and target gene mRNA transcript levels were assessed by RT-qPCR.

### RT-qPCR Analysis

Cells were harvested and total RNA isolated using an RNeasy Kit (Qiagen). Reverse transcription was carried out using Superscript III (Invitrogen) according to the manufacturer's instructions. Quantitative PCR was performed using a Roche Lightcycler 480 II (Roche Diagnostics, Indianapolis, Ind.). Target gene mRNA levels were assessed using gene-specific TaqMan probes (Invitrogen) and dual-color real time PCR method. A β-actin Taqman probe was used as an internal control.

### Example 1

### KDM1A Inhibitors Abrogate the Interaction Between KDM1A and GFI1B

### Displacement of GFI1B from KDM1A/CoREST in SET2 Cells

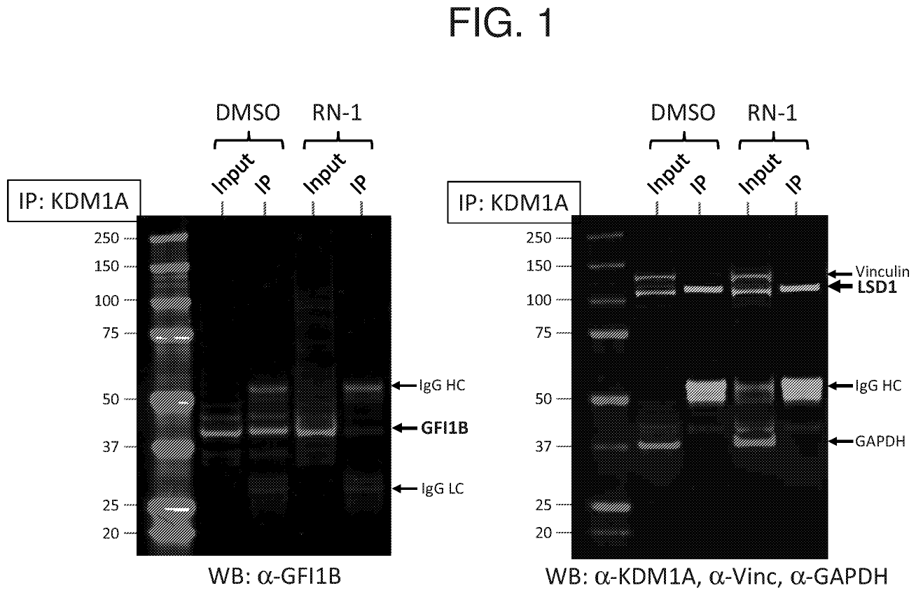

SET2 cells were treated with either DMSO or 1 mM RN-1 for 24 hours. Cell lysates were prepared and an immunoprecipitation was carried out using an anti-KDM1A antibody. SDS-PAGE and Western blotting was performed on the input material and the anti-KDM1A immunoprecipitate. The blots were probed with either an anti-GFI1B antibody (left panel) or with anti-KDM1A, anti-vinculin and anti-GAPDH antibodies (right panel). Bound antibodies were detected with goat anti-rabbit IgG antibody and goat anti-mouse IgG, conjugated with DyLight 800 AND DyLight 680, respectively. Images were generated on a LI-COR Odyssey CLx Imaging System. As shown in FIG. 1, treatment of the SET2 cells with RN-1 resulted in the displacement of GFI1B from the KDM1A/CoREST complex.

### Displacement of GFI1B from KDM1A/CoREST in HEL Cells

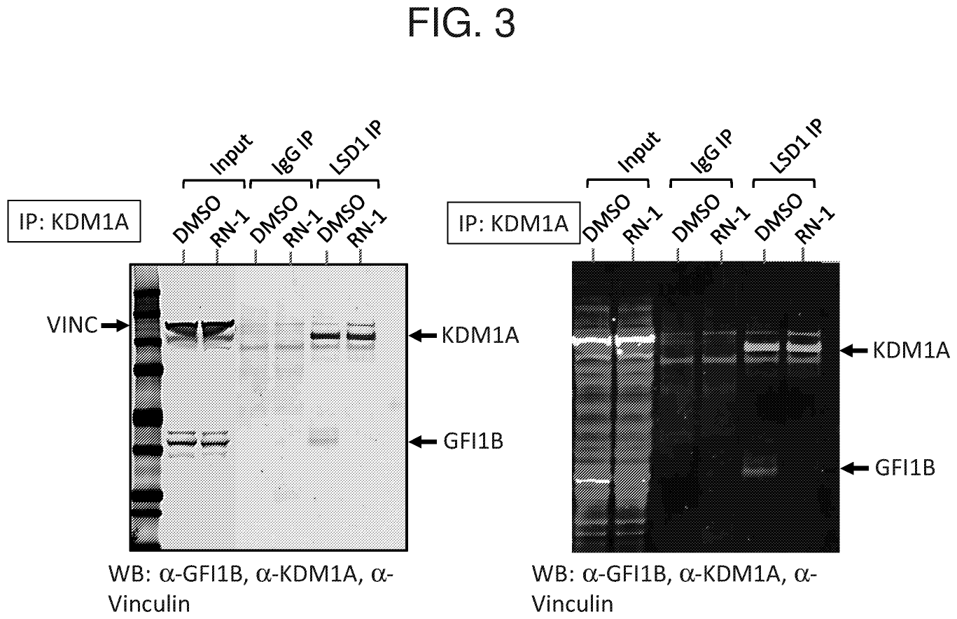

As shown by FIG. 3, treatment of HEL cells (an AML cell line of the M6 subtype, acute erythroid leukemia) also resulted in the displacement of GFI1B from the KDM1A/CoREST complex. Here, HEL cells were treated with the KDM1A inhibitor RN-1 overnight at 1 μM concentration. Cell lysates were prepared and an immunoprecipitation was carried out using an anti-KDM1A antibody and rabbit IgG as a control. SDS-PAGE and Western blotting was performed on the input material and immunoprecipitated material. Blot was probed with an anti-GFI1B, anti-KDM1A, and anti-vinculin antibodies. Bound antibodies were detected with goat anti-rabbit IgG antibody and goat anti-mouse IgG, conjugated with DyLight 800 AND DyLight 680, respectively. Images were generated on a LI-COR Odyssey CLx Imaging System and are shown in FIG. 3.

### KDM1A/CoREST Complex Remains Intact

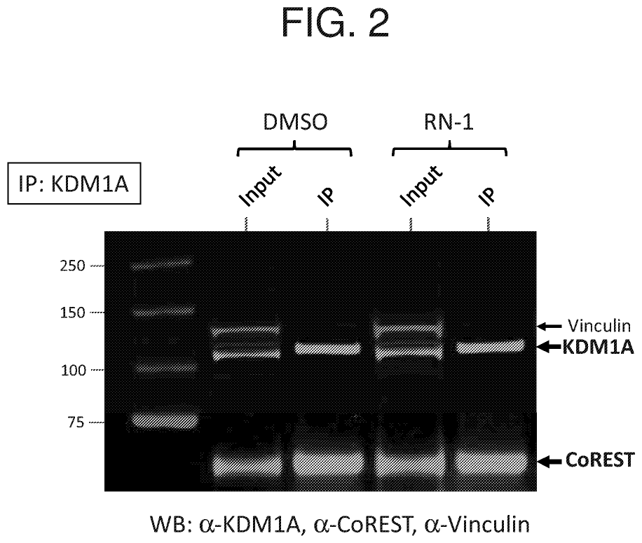

As shown by FIG. 2, CoREST is retained in the KDM1A complex following treatment with RN-1. For this experiment, SET-2 cells were treated with the KDM1A inhibitor RN-1 overnight at 1 μM concentration. Cell lysates were prepared and an immunoprecipitation was carried out using an anti-KDM1A antibody. SDS-PAGE and Western blotting was performed on the input material and the anti-KDM1A immunoprecipitate. The blot was probed with an anti-KDM1A, anti-CoREST, and anti-vinculin antibodies. Bound antibodies were detected with goat anti-rabbit IgG antibody and goat anti-mouse IgG, conjugated with DyLight 800 AND DyLight 680, respectively. Images were generated on a LI-COR Odyssey CLx Imaging System.

In summary, the above experiments show that KDM1A inhibitors, such as RN-1, displace GFI1B from KDM1A/CoREST in SET2 and HEL cells, but do not disassemble the KDM1A/CoREST complex itself. These results support the finding that KDM1A inhibitors selectively abrogate the interaction between KDM1A and GFI1B.

### Example 2

### Cell Lines Expressing Higher Levels of GFI1B are Sensitive to KDM1A Inhibition

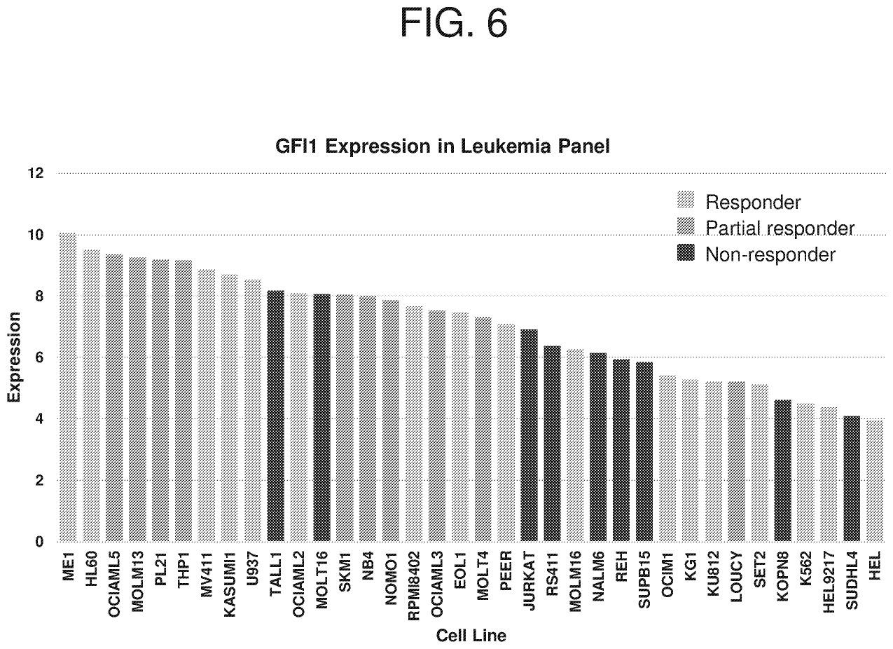

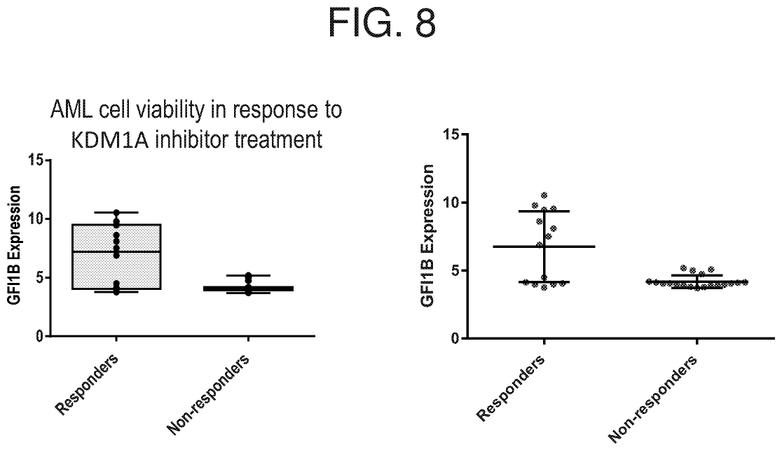

GFI1B expression levels and KDM1A inhibitor response in a panel of human leukemia cell lines is shown in FIG. 4. GFI1B mRNA expression levels were obtained from the Cancer Cell Line Encyclopedia (CCLE) dataset provided by the Broad Institute, MIT. KDM1A inhibitor response was determined in a 12-day cell viability assay using RN-1. Sensitive cell lines were labeled either as Responders (R) with an Eₘₐₓ of greater than 70%, or Partial Responders (PR) with an Eₘₐₓ of less than 70%. Tabular results are shown in FIG. 5. Non-Responders are designated as NR. These data were compared with GFI1 expression levels and KDM1A inhibitor response. See FIG. 6. See also FIG. 8 for an alternative representation of AML leukemia cell lines classified by level of GFI1B mRNA expression, where the left and right panels show a blox and whisker plot.

GFI1 mRNA expression levels were obtained from the Cancer Cell Line Encyclopedia (CCLE) dataset provided by the Broad Institute, MIT. KDM1A inhibitor response was determined in a 12-day cell viability assay using RN-1. Sensitive cell lines were labeled either as Responders with an Eₘₐₓ of greater than 70%, or Partial Responders with an Eₘₐₓ of less than 70%. Tabular results are shown in FIG. 7. Non-Responders are designated as NR.

This data shows that AML cells lines which express higher levels of GFI1B are exquisitely sensitive to KDM1A inhibition. For example, high GFI1B expressors were mostly found across acute myeloid leukemia (AML) M6/M7 FAB subtypes and chronic myeloid leukemia cell lines. See FIG. 5. It was these high GFI1B expressors to which an Eₘₐₓ of greater than 70% was observed, i.e., a responder. This supports the conclusion that cell lines with higher levels of GFI1B (e.g., AML cells) are more sensitive to KDM1A inhibition. Furthermore, high GFI1 expressors were found in subtypes other than AML M6/M7 and in insensitive cell lines (NR). See e.g., FIG. 7. Thus, a correlation exists between GFI1B levels and sensitivity to KDM1A inhibition, but expression of the closely related GFI1 shows no such correlation.

### Example 3

### GFI1B Expression Varies in AML Patient Samples

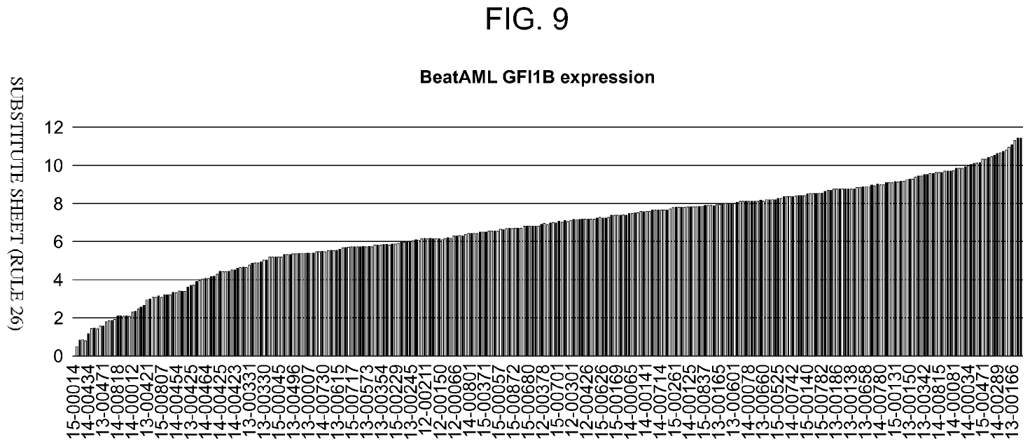

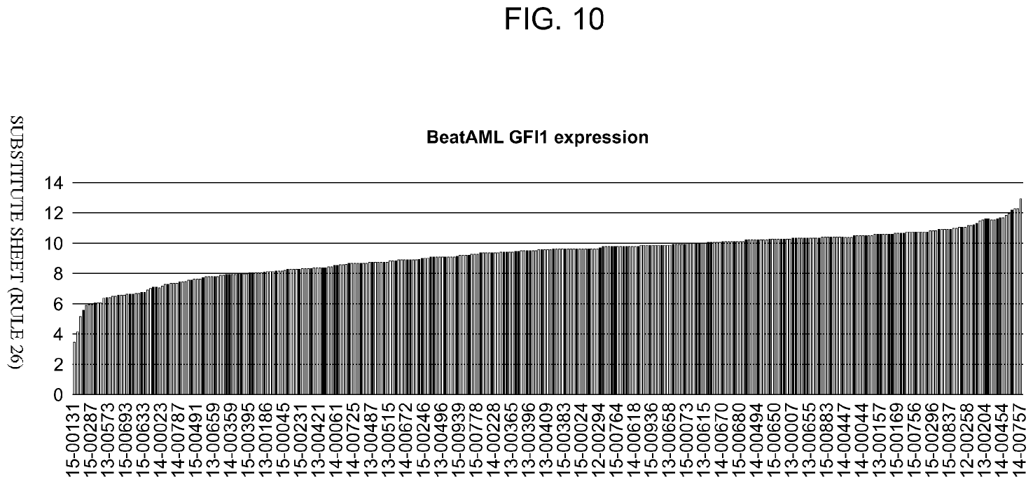

GFI1B and GFI1 mRNA expression was measured in a panel of 325 patient-derived AML samples (bone marrow and/or peripheral blood mononuclear cells. See FIG. 9 and FIG. 10. A large variation of GFI1B expression was found and high GFI1B expression was seen in AML with CCAAT-enhancer binding protein alpha (CEBPA) mutations. See FIG. 9. Conversely, GFI1 expression from samples of 325 AML patients did not vary markedly across the panel. See FIG. 10. These data suggest that GFI1B expression levels can be used as a criterion for selecting a subpopulation of AML patients for treatment with a KDM1A inhibitor because of the broad range in values observed, with a distinct subpopulation that can be characterized as “high” GFI1B expressors.

### GFI1B Knockdown Inhibits Growth of SET2 Cells

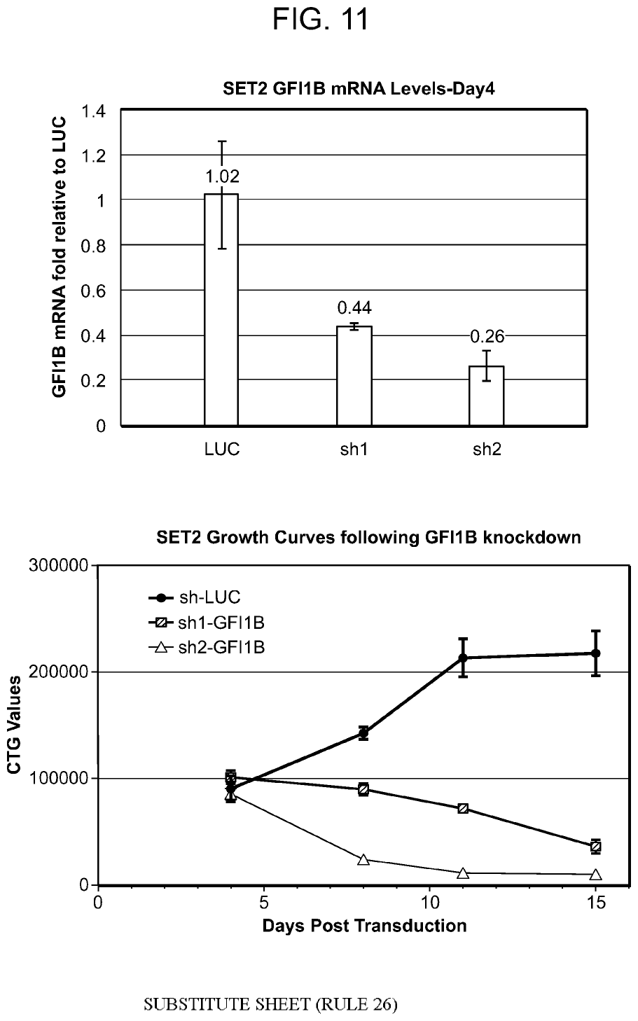

GFI1B mRNA expression levels (left panel) were reduced using shRNAs specifically targeting this gene versus the control shRNA (LUC) targeting the luciferase gene. Growth of SET2 cells was monitored using Cell Titer-Glo (Promega) over the course of 15 days following introduction of the shRNAs. See FIG. 11. As shown, the growth of SET2 cells was inhibited, indicating that GFI1B is essential for tumor cell proliferation.

### Tumor Reduction in Nude Mice

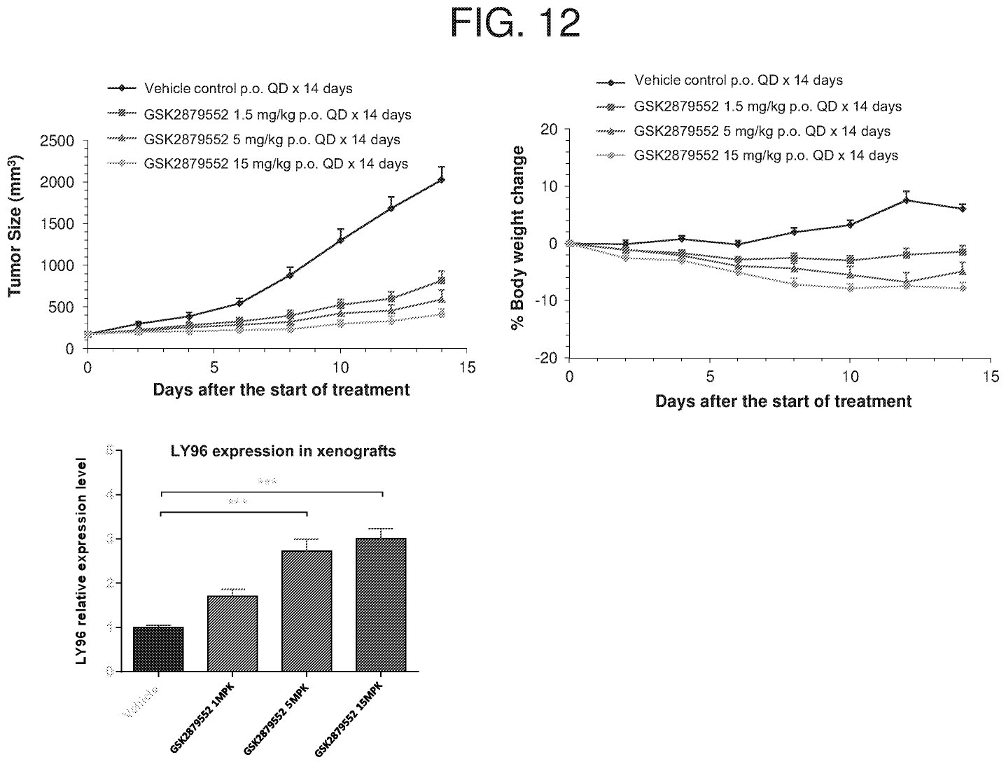

FIG. 12 illustrates the in vivo efficacy of GSK2879552 on SET2 xenografts. Administration of the KDM1A inhibitor GSK2879552 reduced the growth of SET-2 tumor xenografts in Nude mice. Tumor-bearing animals were treated with 3 different dose levels of the KDM1A inhibitor: 1.5 mg/kg, 5 mg/kg, and 15 mg/kg administered orally (p.o.) on a daily schedule (QD). Top left panel shows tumor size as measured over 14 days of dosing. Top right panel indicates body weight changes determined in the treated mice. Bottom panel shows the expression levels of the KDM1A target gene LY96 in the tumor xenografts determined by RT-PCR at the completion of the study.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating a subject with a cancer characterized by a high expression level of GFI1B, comprising administering to the subject a therapeutically effective amount of a KDM1A inhibitor, wherein high expression level of GFI1B is characterized by an expression value falling within the top quartile of measured values from a collection of tissue samples obtained from the same cancer type in a population of subjects.

2. The method of claim 1, wherein prior to treatment, the cancer was determined to have a high expression level of GFI1B.

3. The method of claim 1, comprising the step of performing a biopsy of the subject's cancer prior to treatment and determining if the cancer exhibits a high expression level of GFI1B.

4. A method of treating a subject with a cancer comprising determining the expression level of GFI1B; and administering to the subject a therapeutically effective amount of an KDM1A inhibitor, if the subject's cancer exhibits a high expression level of GFI1B.

5. (canceled)

6. (canceled)

7. The method of claim 4, further comprising administering to the subject a therapeutically effective amount of a cancer therapy other than the administration of a KDM1A inhibitor, if the expression level of GFI1B is not high.

8. The method of claim 1, wherein high expression level of GFI1B is characterized by an expression value falling within the top quartile of measured values from a collection of tissue samples obtained from the same cancer type in a population of subjects.

9. The method of claim 1, wherein the collections of tissue samples are taken from tumors.

10. The method of claim 1, wherein the population of subjects is at least 25 subjects having the same cancer.

11. The method of claim 1, wherein the population of subjects is at least 50 subjects having the same cancer.

12. A method of treating a cancer in a subject, comprising administering to the subject an effective amount of a KDM1A inhibitor and an anti-cancer agent, wherein the cancer is characterized by a GFI1B expression level of between the 1^{st} to 75^{th} percentile of measured values from a collection of tissue samples obtained from the same cancer type in a population of subjects.

13. (canceled)

14. The method of claim 12, wherein prior to treatment, the cancer is characterized by a GFI1B expression level of between the 1^{st} to 75^{th} percentile of measured values from a collection of tissue samples obtained from the same cancer type in a population of subjects.

15. The method of claim 12, wherein the collection of tissue samples are taken from tumors.

16. The method of claim 12, wherein the population of subjects is at least 25 subjects having the same cancer.

17. The method of claim 12, wherein the population of subjects is at least 50 subjects having the same cancer.

18. The method of claim 1, wherein the cancer is selected from breast cancer, colorectal cancer, pancreatic cancer, cervical cancer, T cell lymphoma, uveal melanoma, gastric carcinoma, colorectal carcinoma, ovarian carcinoma, hepatocellular carcinoma, melanoma, glioma, cardiac, gastrointestinal, genitourinary tract, liver, bone, nervous system, gynecological, hematologic, skin, and adrenal cancers.

19. (canceled)

20. The method of claim 1, wherein the cancer is selected from small cell lung carcinoma, neuroblastoma, Merkel cell carcinoma, myeloproliferative diseases, and myelodysplastic syndrome.

21. The method of claim 1, wherein the cancer is acute myeloid leukemia or chronic myeloid leukemia.

22. (canceled)

23. (canceled)

24. The method of claim 1, wherein the KDM1A inhibitor is selected from 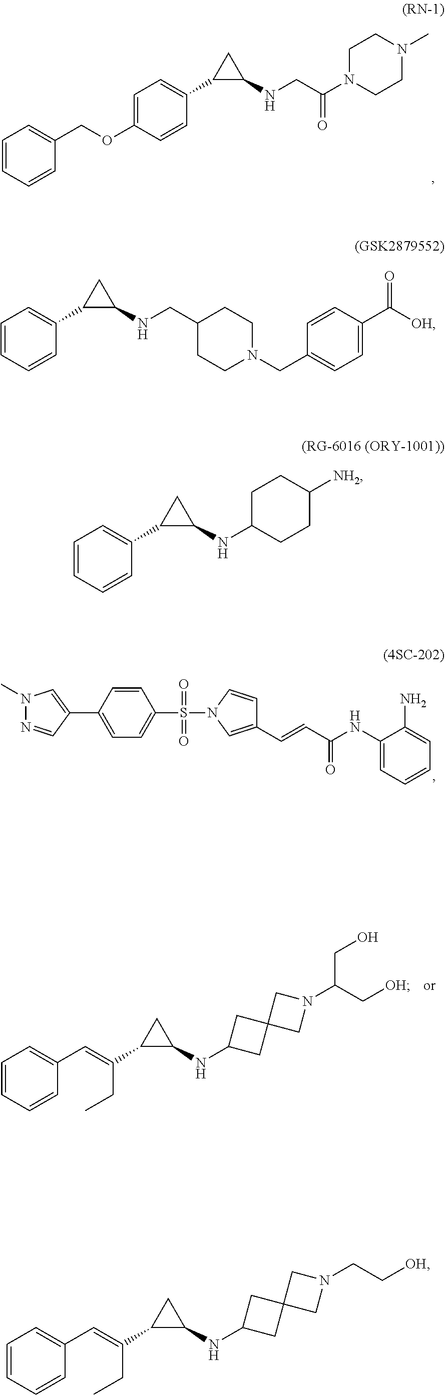
or a pharmaceutically acceptable salt thereof.

25. The method of claim 1, wherein the KDM1A inhibitor is 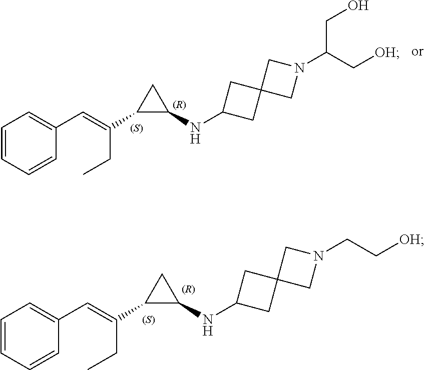
or a pharmaceutically acceptable salt thereof.

26. The method of claim 1, wherein the KDM1A inhibitor is 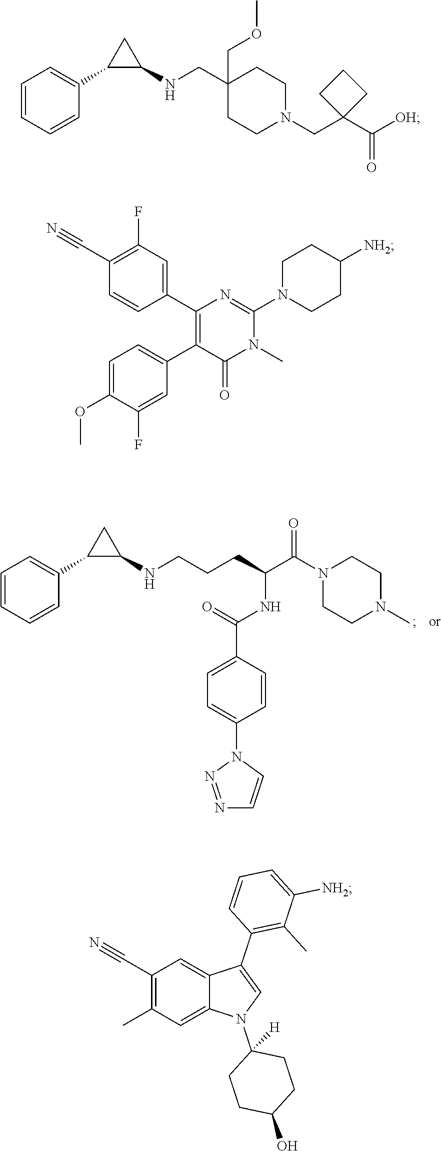 [SMILES: COCC1(CN[C@@H]2C[C@H]2c2ccccc2)CCN(CC2(C(=O)O)CCC2)CC1 ; COc1ccc(-c2c(-c3ccc(C#N)c(F)c3)nc(N3CCC(N)CC3)n(C)c2=O)cc1F ; CN1CCN(C(=O)[C@H](CCCN[C@@H]2C[C@H]2c2ccccc2)NC(=O)c2ccc(-n3ccnn3)cc2)CC1 ; Cc1cc2c(cc1C#N)c(-c1cccc(N)c1C)cn2[C@H]1CC[C@@H](O)CC1]
or a pharmaceutically acceptable salt thereof.

27-33. (canceled)
